(19) United States
(12) Patent Application Publication
(10) Pub. No.: US 2005/0119504 A1
(43) Pub. Date: Jun. 2, 2005

(54) New process for the preparation of a biphenyl ether compound

(75) Inventors: Christopher Paul Ashcroft, County of Kent (GB); Lynsey Charlotte Hesmondhalgh, County of Kent (GB); Iain Robert Gladwell, County of Kent (GB); Laurence James Harris, County of Kent (GB); Michael Leslie Hughes, County of Kent (GB); Juergen Thomas Klotz, County of Kent (GB); Philip Charles Levett, County of Kent (GB); Matthew Charles Morland, County of Kent (GB); Nicholas Murray Thomson, County of Kent (GB)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/945,001

(22) Filed: Sep. 20, 2004

Related U.S. Application Data
(60) Provisional application No. 60/583,925, filed on Jun. 29, 2004.

(30) Foreign Application Priority Data
Sep. 22, 2003 (GB) 0322150.4
Jun. 14, 2004 (GB) 0413229.6

(51) Int. Cl. C07C 311/18

(52) U.S. Cl. 562/585; 564/86

(57) ABSTRACT

The present invention is concerned with an improved process for the preparation of the selective serotonin reuptake inhibitor 3-[(Dimethylamino)methyl]-4-[4-(methylsulfanyl)phenoxy]benzenesulfonamide (L) or (D) tartrate (I) and with intermediate products therein.

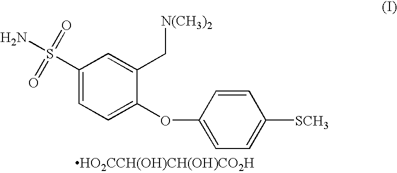

This application claims the priority from United Kingdom Application Number 0322150.4, filed on Sep. 22, 2003, United Kingdom Application Number 0413229.6, filed on Jun. 14, 2004, and the benefit from U.S. application Ser. No. 60/583,925, filed on Jun. 29, 2004.

The present invention is concerned with an improved process for the preparation of the selective serotonin reuptake inhibitor 3-[(Dimethylamino)methyl]-4-[4-(methylsulfanyl)phenoxy]benzenesulfonamide (L) or (D) tartrate and with intermediate products therein.

WO01/72687 describes the preparation of 3-[(Dimethylamino)methyl]-4-[4-(methylsulfanyl)phenoxy]benzenesulfonamide (L) tartrate (I) in which the compound is prepared by (i) reacting 4-(methylmercapto)phenol (III) with 2-fluorobenzaldehyde (II) in the presence of potassium carbonate in a suitable solvent such as DMF; (ii) carrying out a reductive amination of 2-[4-(Methylsulfanyl)phenoxy]benzaldehyde (IV) with Sodium triacetoxyborohydride and dimethylamine hydrochloride and then optionally forming the HCl salt of the product; (iii) reacting N,N-Dimethyl-N-{2-[4-(methylsulfanyl)phenoxy]benzyl}amine (V) with chlorosulfonic acid in dichloromethane; and (iv) treating 3-[(Dimethylamino)methyl]-4-[4-(methylsulfanyl )phenoxy]benzenesulfonyl chloride (VI) with aqueous ammonia or ammonia in ethanol to give (VII). Steps (iii) and (iv) may be combined. The corresponding tartrate salt may be obtained by dissolving (VII) in an organic solvent, adding the appropriate tartaric acid, optionally cooling the solution and collecting the resulting crystals of (I).

The complete sequence may be represented as follows:

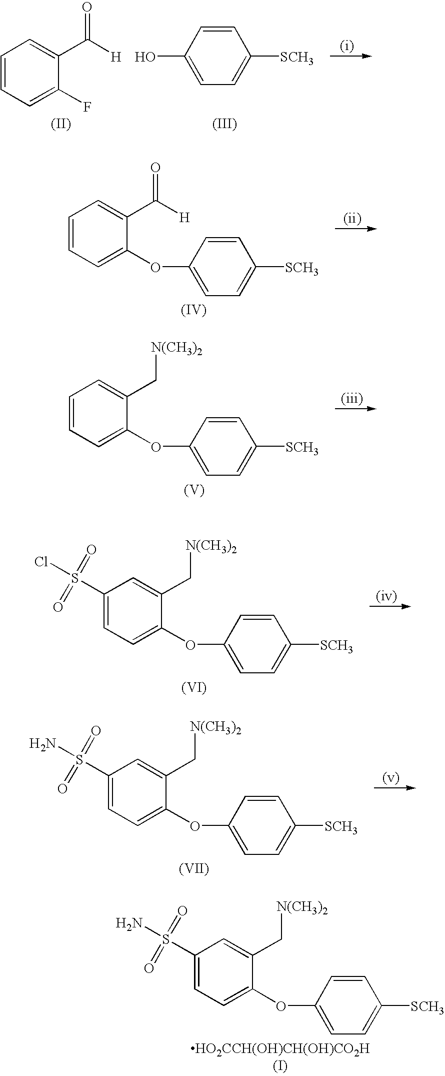

There are a number of problems with this route:
(a) Process step (i) is carried out under dilute reaction conditions, the result of which is that a large amount of waste solvent must be disposed of at the end of the reaction. Additionally, when the reaction is carried out on a large scale reaction times are slow. Furthermore, the product of the reaction, compound (IV), is difficult to isolate. It has a low melting point (37-39° C.), and therefore the compound is not amenable to drying in a vacuum oven, which makes it difficult to remove the solvent from the product at the end of the reaction. Isolation by crystallisation is also hindered by this property.
(b) The reductive amination of compound (IV), process step (ii), proceeds slowly, particularly on a larger scale where reactions can take up to 1 week to reach completion, this has significant economic disadvantages. Furthermore the yields are modest and impurities are generated. The generation of by-products, such as the primary alcohol derivative of compound (IV), reduces the yield further.
(c) In process step (iii), the chlorosulfonylation of compound (V) is carried out using a large excess of 97% chlorosulfonic acid (10 molar equivalents) in the solvent dichloromethane. The hazardous nature of the reagent and solvent makes them difficult to handle safely, particularly on a large scale. Furthermore the excess reagent must be neutralised at the end of the reaction generating a large amount of waste. The disposal of large volumes of dichloromethane is also expensive and harmful to the environment. Additionally, several impurities are generated as by-products in this process step. These impurities have to be carried through to the next step of the sequence due to the highly reactive nature of compound (VI) and its physical form (a sticky solid), which render it difficult to isolate and purify effectively.
(d) Due to the moderate purity of compound (VI), process step (iv) is low yielding. Additionally, the sulfonic acid derivative (IX) is generated as a by-product. Compound (IX) is difficult to separate from the desired product, compound (VII), as are the impurities carried through from process step (iii).

In summary, whilst this reaction sequence provides an adequate route for the production of compounds of formula (I) on a laboratory scale, there is a clear requirement for a robust process that would be more applicable to industrial scale generation of these compounds.

As a result an improved synthetic process has been developed to 3-[(Dimethylamino)methyl]-4-[4-(methylsulfanyl)phenoxy]benzenesulfonamide (L) or (D) tartrate (I) which overcomes the problems described above.

The complete sequence may be represented as follows:

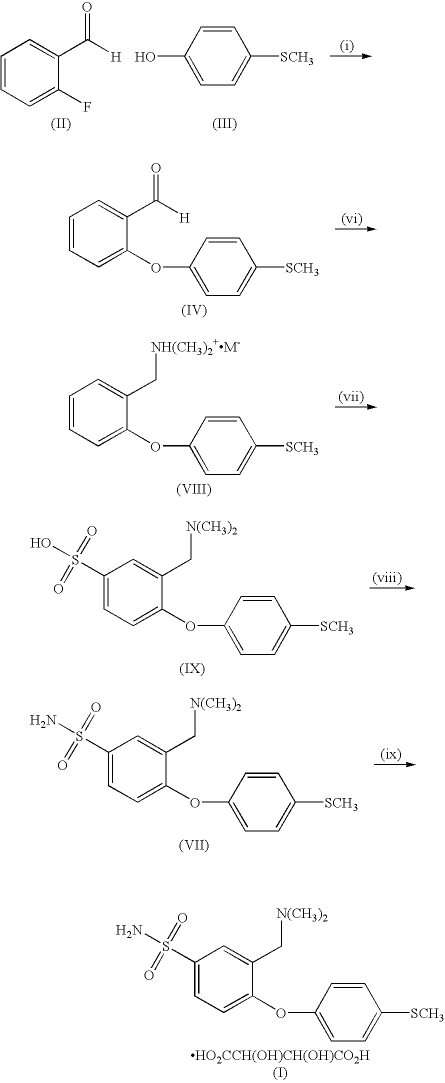

In one embodiment of the invention compounds of formula (IV) may be prepared by reacting compounds of formula (II) and (III) under the conditions of process step (i), nucleophilic aromatic substitution, in the presence of a base in a suitable solvent.

Suitable bases include carbonate bases such as potassium carbonate, sodium carbonate, caesium carbonate; butoxide bases such as potassium t-butoxide, lithium t-butoxide, sodium t-butoxide; hydroxide bases such as sodium hydroxide; and organic bases such as pyridine and morpholine.

Suitable solvents include polar aprotic solvents such as N,N-dimethylformamide, tetrahydrofuran, dimethylsulfoxide, dioxan, acetonitrile and ethers.

The preferred base for the reaction is potassium carbonate and the preferred solvent is N,N-dimethylformamide.

Most preferably the potassium carbonate is of small particle size (D₉₀<1000 microns).

The resultant compounds of formula (VIII) may be prepared by process step (vi), a reductive amination reaction, by reacting compounds of formula (IV) with a dimethylamine source and a suitable reducing agent; wherein M is a suitable counter-ion such as chloride, bromide, toluenesulfonate, benzenesulfonate, methane sulfonate, hydrogen sulfate, acetate or trfluororacetate.

Suitable sources of dimethylamine include dimethylamine, salts of dimethylamine in the presence of a base (a suitable salt would include hydrochloride, a suitable base would include triethylamine); and N,N-dimethylformamide in the presence of acid or base (a suitable acid would include formic acid; a suitable base would include triethylamine).

Suitable reducing agents include sodium borohydride, sodium triacetoxyborohydride, sodium cyanoborohydride, hydrogen gas in the presence of a catalyst, formic acid and formic acid salts such as potassium formate and sodium formate.

In some cases addition of a Lewis acid such as titanium tetra-iso-propoxide may be beneficial.

Suitable solvents for the reaction include dichloromethane, tetrahydrofuran, tert-butylmethylether, ethanol, ethylacetate, N,N-dimethylformamide.

The preferred source of reducing agent is formic acid wherein the required dimethylamine is generated by the acid-mediated degradation of N,N-dimethylformamide.

N,N-dimethylformamide is the preferred solvent for the reaction.

The reaction is preferably carried out at elevated temperature.

The intermediate tertiary amine product may then be isolated as a crystalline salt by reacting the amine with a suitable acid in the presence of a suitable solvent.

Suitable acids include hydrochloric acid, hydrobromic acid, sulfuric acid, methanesulfonic acid, toluene sulfonic acid, benzenesulfonic acid, acetic acid and trifluororacetic acid.

Preferred acids are hydrochloric acid, sulfuric acid and methanesulfonic acid.

Suitable solvents include tert-butylmethylether and methylethylketone, either alone, in combination or in the presence of some water.

Sulfuric acid salts are particularly preferred. Preferred conditions for their preparation are treatment with methyl ethyl ketone and sulfuric acid.

Process steps 1 and 2 may be combined, that is that compounds of formula (IV) are not isolated and purified. This is particularly advantageous as the low melting point of compound (IV) makes it particularly difficult to isolate.

Accordingly in one embodiment of the invention compounds of formula (VIII) may be prepared by reacting compounds of formula (II) and (III) under the conditions of process step (i), before treating the crude reaction mixture under the conditions of process step (vi).

In this embodiment, preferred conditions for process step (i) are N,N-dimethyformamide as solvent and potassium carbonate as base.

Most preferably the potassium carbonate is of small particle size (D₉₀<1000 microns).

In this embodiment, preferred conditions for process step (vi) are N,N-dimethylformamide as solvent and formic acid as reducing agent, at elevated temperature.

According to another embodiment of the invention Compounds of formula (IX) may be prepared by process step (vii), a sulfonylation reaction by reacting compounds of formula (VIII) in the presence of a sulfonylating reagent in the presence of a suitable solvent.

Suitable sulfonylating reagents include chlorosulfonic acid, sulfuric acid and fuming sulfuric acid.

The preferred sulfonylating agent is chlorosufonic acid (99%).

Suitable solvents include dichloromethane, chlorosulfonic acid, trifluoroacetic acid, methanesulfonic acid and sulfuric acid.

Preferred solvents are trifluoroacetic acid and methanesulfonic acid.

Most preferred conditions are chlorosulfonic acid (99%) and methanesulfonic acid or chlorosulfonic acid (99%) and trifluoroacetic acid.

The preferred reaction temperature is between 0-5° C., when trifluoroacetic acid is the solvent. The preferred reaction temperature is 0° C. to room temperature, when methansulfonic acid is the solvent.

According to yet another embodiment of the invention Compounds of formula (VII) may be prepared by process step (viii), formation of a sulfonamide by reacting compounds of formula (IX) with a chlorinating agent in a suitable solvent, before quenching the sulfonyl chloride intermediate with ammonia.

Suitable chlorinating agents include PCl₅, POCl₃, SOCl₂ and (COCl)₂.

Suitable solvents include acetonitrile, propionitrile, toluene and ethylacetate.

Suitable ammonia sources include ammonia gas and a solution of ammonia gas in either an organic solvent or water.

Preferred conditions include Phosphorus oxychloride in acetonitrile followed by treatment with aqueous ammonia.

Most preferred conditions encompass the addition of the aqueous ammonia to a solution of the intermediate sulfonylchloride (VI), followed by treatment with water.

In a further embodiment of the invention, the resultant compound of formula (VII) may be treated with absorbents to enhance its purity. Suitable absorbents include activated charcoal, resins and Fuller's Earth.

According to yet another embodiment of the invention Compounds of formula (I) may be prepared by process step (ix), by reacting compounds of formula (VII) with D or L Tartaric acid in a solvent system.

Suitable solvent systems include iso-propyl alcohol, iso-propyl alcohol/water, Ethanol, ethanol/water, methyl ethyl ketone, methyl ethyl ketone/water, methyl iso-butyl ketone, methyl iso-butyl ketone/water, acetone, acetone/water

Most preferred conditions are aqueous (L)-Tartaric acid with methyl ethyl ketone as solvent.

Formation of the tartrate salt using the above solvent system gives salts of improved purity in improved yield in a process suitable for an industrial scale.

The advantages of this new process may be summarised as follows:
i) The use of potassium carbonate of particle size D₉₀<1000 microns, reduces the reaction time required for process step (i) to reach completion when carried out on a large scale. The use of this reagent facilitates the completion of multi-kilo reactions in less than 24 hours; this has significant economic advantages.
ii) The use of N,N-dimethylformamide as both solvent and dimethylamine source in process step (vi) allows the combination of process steps (i) and (vi) to be possible. The combination of process steps (i) and (vi) avoids the isolation of low melting intermediate compound (IV) and also significantly reduces the volume of solvent required for these two transformations. Reaction efficiency is thus increased as more product is generated from a smaller reaction volume.
iii) The use of formic acid as reducing agent in process step (vi) is particularly advantageous for two reasons. Firstly, as it is a liquid, it can be added in a controlled fashion to the reaction mixture at the end of process step (i). Therefore the gas evolution that accompanies the pH change between process steps (i) and (vi) can be managed safely. Secondly, the oxidation of formic acid does not generate any chemical waste; CO₂ is the sole by product.
iv) The purity of compound (VIII) is increased by the formation of its preferred sulphate salt during process step (vi). This salt form is easily isolated from the reaction mixture at the end of the reaction.
v) In process step (vii), use of 99% chlorosufonic acid (instead of the 97% chlorosufonic acid used in process step (iii)) reduces the amount of by-products generated by more than 50%. Additionally far less sulfonylating reagent is required as compared to process step (iii), therefore there is less chemical waste to dispose of at the end of the reaction. Furthermore, the solvent dichloromethane can be replaced by the more environmentally benign methanesulfonic acid, or trifluoroactectic acid.
vi) Compound (IX) is formed as a precipitate from process step (vii). The ability to isolate this product provides a valuable opportunity to purify this intermediate, if necessary, at the mid-point of the reaction sequence. This allows greater control to be exercised with respect to purity during the process.
vii) The isolation of reactive intermediate compound (VI) is avoided by process step (viii) where it is generated in situ. The reaction conditions employed in this step are milder than those of reaction step (iii) and therefore fewer impurities are generated, improving the purity of the intermediate.
viii) The subsequent quenching of this intermediate is carried out in a novel fashion by the addition of aqueous ammonia to the reaction mixture, followed by the addition of water and carrying out an in situ reflux. Accepted industrial scale practice in the art would be to add the reaction mixture to an excess of aqueous ammonia. This new, reversed mode of addition has the following advantages:
   (a) The by-product sulfonic acid derivative (IX) is soluble in the reaction mixture which facilitates its removal from the product.
   (b) It is not necessary to transfer the slurry of intermediate compound (VI) to another reaction vessel, thereby avoiding the handling problems associated with its physical form.
   (c) The reflux increases the solubility of the inorganic impurities in the reaction mixture, and therefore aids their separation from the product. In addition, organic impurities are purged.

The above advantages result in a higher yield of compound (VII).
ix) This new process is suitable for producing quantities of compound (I) on an industrial scale.

In a further embodiment of the present invention are provided compounds of formula (VIII) and (IX) wherein M is a suitable counter-ion as described above.

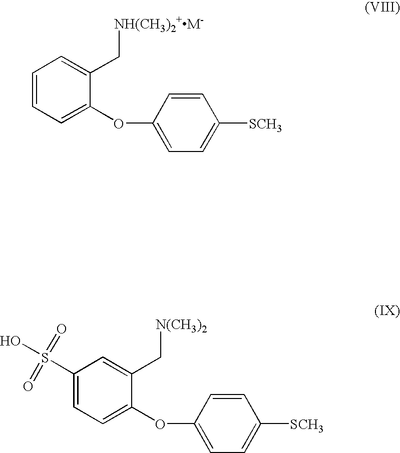

These compounds are particularly useful in the synthesis of compounds of formula (I) by the process of the present invention.

Experimental

The following abbreviations and definitions are used:

| | |
|---|---|
| MEK | methylethyl ketone |
| DMF | N,N-dimethylformamide |
| TBME | tertiary-butyl methyl ether |
| DMSO | dimethylsulfoxide |
| POCI₃ | phosphorousoxychloride |
| DCM | dichloromethane |
| DMSO | dimethylsulfoxide |
| m/z | mass spectrum peak |
| HPLC | High Pressure Liquid Chromatography |
| MS | mass spectrum |
| NMR | nuclear magnetic resonance |
| q | quartet |
| s | singlet |
| t | triplet |
| br | broad |
| TFA | trifluoroacetic acid |
| MSA | Methanesulfonic acid |
| Kg | Kilograms |
| L | Litre |
| mL | millilitre |
| g | grams |
| CDCl₃ | deuterated chloroform |

The powder X-ray diffraction (PXRD) patterns were determined using a Siemens D5000 powder X-ray diffractometer fitted with a theta-theta goniometer, automatic beam divergence slits, a secondary monochromator and a scintillation counter. The specimen was rotated whilst being irradiated with copper K-alphal X-rays (Wavelength=1.5046 Angstroms) filtered with a graphite monochromator (λ=0.15405 nm) with the X-ray tube operated at 40 kV/40 mA. The main peaks (in degrees 2 θ) of the PXRD patterns for the various solid forms are illustrated.

Melting points were determined using either a Perkin Elmer DSC7 at a heating rate of 20° C./minute or a Buchi melting point B-545.

NMR spectra were obtained using a Varian Inova 300 MHz spectrometer by dissolving the sample in an appropriate solvent.

Mass spectra were obtained using a LC/MS system consisting of an 1100 series Hewlett Packard LC in combination with a Micromass ZMD mass spectrometer.
N,N-dimethyl-2-[4-(methylthio)phenoxy]benzylaminehydrogen sulfate

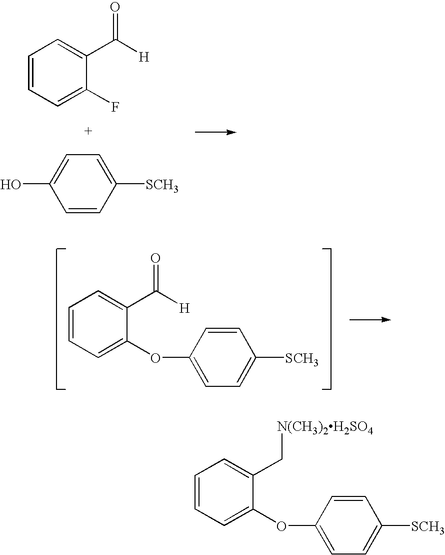

2-fluorobenzaldehyde (38.0 Kg), 4-(methylmercapto)phenol (43.8 Kg), potassium carbonate (46.6 Kg, with a particle size D₉₀<1000 microns) and DMF (171 L) were charged to a suitable reactor and heated to 110° C. for 24 hours. When all the 2-fluorobenzaldehyde was consumed (<3% as evidenced by HPLC) the reaction mixture was cooled to room temperature, and treated with formic acid (169.1 Kg) over 30 mins. The mixture was heated to 130° C. for a further 24 hours and then allowed to cool to room temperature. Water (9.5 L) was added followed by conc. aqueous ammonia (152 L) to adjust the pH to greater than 8.5. The mixture was extracted with TBME (114 L) and the phases allowed to separate, the lower aqueous phase was then discarded. To prepare the sulfate salt the TMBE extract was diluted with MEK (114 L). The solution was cooled to 15° C. and concentrated sulphuric acid (30.6 Kg) was added keeping the temperature below 25° C. The mixture was then allowed to cool to 20° C. and stirred overnight, finally the mixture was cooled to 0-5° C. for 1 hour and the product collected by filtration at reduced pressure. The filter cake was washed with MEK (76 L). The product was then dried at 50° C. under vacuum overnight. Yield=81%, δ_{H} (DMSO-d₆, 300 MHz) 2.48 (6H, s), 2.81 (3H, s), 4.39 (2H, s), 6.82 (1H, d), 7.05 (2H, d), 7.22 (1H, t), 7.39 (2H, d), 7.43 (1H, t), 7.61 (1 H, d), 9.46 (1H, br, s); MS m/z (TS⁺) 274 (MH⁺), Melting point=139-141° C.
3-[(dimethylamino)methyl]-4-[4-(methylthio)phenoxy]benzenesulfonic acid

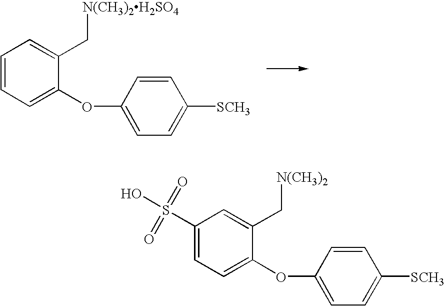

The title compound may be prepared either using methanesulfonic acid (method A) or trifluoroacetic acid (method B) as solvent.

Method A.

To a suitable vessel methanesulfonic acid (17.66 L) was charged followed by N, N-dimethyl-2-[4-(methylthio)phenoxy]benzylaminehydrogen sulfate (7.85 Kg), the mixture was stirred at room temperature until a solution was achieved. The reaction mixture was cooled to 0° C. and treated with chlorosulfonic acid (11.36 Kg), keeping temperature below 5° C., over 1 hour. The reaction was monitored by HPLC and the reaction was complete after 5 hours with <2% starting material detected. In a separate vessel water (70.65 Kg) was cooled to 5° C. The cooled reaction mixture was then quenched into the cooled water keeping the temperature below 35° C. A thick white precipitate was formed during the quench. Finally wash the remaining reaction mixture into the quench with methanesulfonic acid (2.91 Kg), then water (7.85 Kg). The resultant slurry was stirred overnight at room temperature before cooling to 0° C. for 1 hour. The product was filtered under reduced pressure and the cake washed with water (15.7 L). The solid product was then stirred with water (78.5 L) at room temperature for 1 hour. The product was filtered at reduced pressure and the cake washed with water (15.7 L). The material was then dried at 50° C. under vacuum overnight. Yield=62%.

Method B.

To a suitable vessel trifluoroacetic acid (138 mL) was charged followed by N,N-dimethyl-2-[4-(methylthio)phenoxy]benzylaminehydrogen sulfate (50 g), the mixture was stirred at −3° C. until a solution was achieved. The reaction mixture was maintained at −3° C. and treated with chlorosulfonic acid (36 mL) while keeping the temperature below 6° C., over 0.25 hour. Using 99% grade chlorosulfonic acid minimises impurity generation in the reaction (compared to lower grades of reagent) and the resulting solid is therefore isolated with increased purity. The reaction was monitored by HPLC and the reaction was complete after 24 hours with <2% starting material detected. In a separate vessel water (500 mL) was cooled to 2° C. The reaction mixture was then quenched into the cooled water (over 2.5 mins) keeping the temperature below 27° C. The fast addition is necessary to keep the product in solution until the end of the addition, at which time the product begins to slowly precipitate out, and the largest crystal size is observed. The reaction mixture was washed into the quenched mixture with trifluoroacetic acid (12 mL) and the slurry was stirred at 20° C. for 2.5 hour, and then at 0° C. overnight. The product was filtered under reduced pressure.

Several options are available for upgrading the quality of the material before drying.

Option 1 The solid product was stirred in water (250 mL) at room temperature for 0.5 hour and then filtered at reduced pressure. The solid was stirred in a 1:1 mixture of acetonitrile/water (250 mL) for 2 hours at 40° C. The slurry was cooled to room temperature and after 1 hour, filtered under reduced pressure. The 1:1 acetonitrile/water (250 mL) reslurry at 40° C. was repeated once more on the damp product and then dried at 50° C. under vacuum overnight. Yield=54%.

Option 2 The solid product was washed with water (2×50 mL). The solid was then stirred in a 1:1 mixture of acetonitrile/water (250 mL) at 60° C. for 1 hour. The slurry was cooled to room temperature and stirred at this temperature for 4 hours. The product was filtered under reduced pressure and then dried at 50° C. under vacuum overnight. Yield=55%.

Option 3 The solid was then stirred in a 1:1 mixture of acetonitrile/water (250 mL) at 60° C. for 1 hour. The slurry was cooled to room temperature and stirred at this temperature for 4 hours. The product was filtered under reduced pressure and then dried at 50° C. under vacuum overnight. Yield=58%.

To further upgrade the purity of this key intermediate an additional reslurry or recrystallisation may be carried out if necessary. The recrystallisation improves purity to a greater extent than the reslurry and the process is outlined below.

Acetonitrile (24.9 L), water (20.75 L) and 3-[(dimethylamino)methyl]4-[4-(methylthio)phenoxy]benzenesulfonic acid (4.15 Kg) was charged to a vessel and heated to reflux for 1 hour. The resultant solution was then cooled to room temperature over 3 hours and the slurry was stirred overnight at that temperature. The solid was collected by filtration at reduced pressure and the cake was washed with a 1:1 mixture of acetonitrile and water (4.15 L of each). The material was then dried at 50° C. under vacuum overnight. Yield=72%, δ_{H} (DMSO-d₆, 300 MHz) 2.52 (3H, s), 2.80 (6H, s), 4.40 (2H, d), 6.78 (1H, d), 7.04 (2H, d), 7.18 (2H, d), 7.62 (1H, d), 7.93 (1H, s), 9.55 (1H, br, s); MS m/z (TS⁻) 352 (MH⁻).
3-[(Dimethylaminomethyl]-4-[4-(methylthio)phenoxy]benzenesulfonamide.

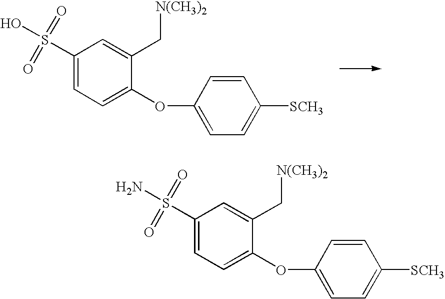

Acetonitrile (60 mL) was charged to a vessel and 3-[(dimethylamino)methyl]-4-[4-(methylthio)phenoxy]benzenesulfonic acid (10.0 g) was added followed by POCl₃ (2.9 mL). The reaction mixture was heated to reflux (approx. 81° C.) for 2 hours. The reaction was monitored by HPLC and was deemed to be complete when starting material was reduced to <2%. The reaction mixture was then cooled to −10° C. and treated with concentrated aqueous ammonia (60 mL) keeping the temperature below 20° C. The reaction mixture was then treated with further water (60 mL) at 40° C. Here an optional heat cycle to reflux for 1 hour can be used before cooling to room temperature. The optional heat cycle affords a higher level of purging of process related impurities. The reaction mixture was stirred overnight at room temperature. The resultant solid was filtered at reduced pressure and the filter cake washed with water (20 mL) before being dried at 50° C. under vacuum overnight. Yield=88%. δ_{H} (CDCl₃, 300 MHz) 2.35 (6H, s), 2.49 (3H, s), 3.66 (2H, s), 5.20 (2H, br), 6.81 (1H, d), 6.92 (2H, d), 7.27 (2H, d), 7.72 (1H, dd), 8.14 (1H, d); MS m/z (TS⁺) 353 (MH⁺).
3-[(dimethylamino)methyl]-4-[4-methylthio)phenoxy]benzenesulfonamide (R,R)-tartrate.

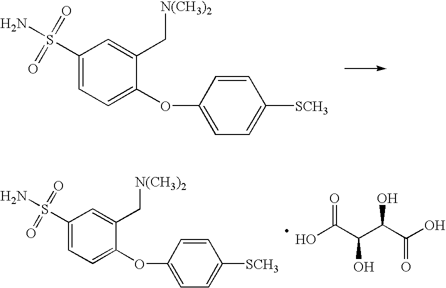

There are two options for preparing 3-[(dimethylamino)methyl]-4-[4-methylthio)phenoxy]benzenesulfonamide (R,R)-tartrate depending on whether activated carbon is used in a loose or solid supported form.

Option 1

3-[(Dimethylamino)methyl]-4-[4-(methylthio)phenoxy]benzenesulfonamide (10 g) was mixed with MEK (80 mL) at room temperature. The stirred mixture was heated to reflux (approx. 80° C.) for 15 minutes and then cooled to room temperature. The mixture was treated with activated carbon (20% w/w, 2 g of Cuno ‘Pfizer Type A’). The suspension was stirred at room temperature for 15 minutes and then filtered washing the carbon with a further amount of MEK (20 mL). The MEK solution was treated with a solution of (L)-tartaric acid (4.26 g) dissolved in water (13 mL) and MEK (13 mL) at room temperature over 10 minutes and the resultant slurry was stirred at room temperature for 1 hour. The solid product was then collected by filtration at reduced pressure and the filter cake was washed with MEK (20 mL). The salt was dried at 50° C. under vacuum. Yield=82%.

Option 2

3-[(Dimethylamino)methyl]-4-[4-(methylthio)phenoxy]benzenesulfonamide (31.65 Kg) was mixed with MEK (253.2 L) at room temperature. The stirred mixture was heated to reflux (approx. 80° C.) for 1 hour and then cooled to room temperature. The mixture was clarified by filtration at reduced pressure. The solution was passed through a solid supported activated carbon cartridge at 10-12 L/minute (0.5 g/cm² carbon surface area, of Cuno ‘R50Sp Pfizer Type A’). MEK (95 L) was used to wash the filter cake followed by the solid supported carbon cartridge. The MEK solution was treated with a solution of (L)-tartaric acid (13.5 Kg) dissolved in water (41.0 L) and MEK (41.1 L) at room temperature over 20 minutes, washing in with a further amount of water (16 L) and the resultant slurry was stirred at room temperature for 3 hours. The solid product was then collected by filtration at reduced pressure and the filter cake was washed with MEK (63.3 L). The salt was then dried at 50° C. under vacuum. Yield=87%.

The main peaks (in degrees 2 θ) of the PXRD pattern are as follows:

| Angle 2-Theata ° | Intensity % |
|---|---|
| 4.330 | 21.9 |
| 12.165 | 5.6 |
| 12.425 | 10.1 |
| 12.543 | 5.4 |
| 13.218 | 12.7 |
| 14.368 | 6.3 |
| 14.463 | 6.3 |
| 16.849 | 7.3 |
| 17.149 | 57.2 |
| 17.469 | 49.5 |
| 17.623 | 66.1 |
| 18.498 | 9.5 |
| 19.403 | 47.2 |
| 20.422 | 12.4 |
| 20.733 | 15.7 |
| 20.923 | 28.2 |
| 21.914 | 100.0 |
| 23.542 | 19.0 |
| 23.776 | 20.0 |
| 24.958 | 30.8 |
| 25.156 | 12.0 |
| 25.787 | 6.1 |
| 26.057 | 12.4 |
| 26.114 | 14.5 |
| 26.408 | 11.6 |
| 26.642 | 25.5 |
| 26.830 | 25.4 |
| 27.130 | 26.7 |
| 27.540 | 12.6 |
| 28.001 | 20.7 |
| 29.122 | 17.7 |
| 29.772 | 28.7 |
| 30.394 | 13.5 |
| 30.983 | 12.8 |
| 31.259 | 32.9 |
| 32.085 | 14.6 |
| 32.258 | 9.5 |
| 32.818 | 5.2 |
| 33.433 | 6.5 |
| 34.085 | 20.3 |
| 34.663 | 6.7 |
| 35.071 | 5.6 |
| 35.674 | 15.2 |
| 35.788 | 14.7 |
| 36.228 | 10.7 |
| 36.517 | 8.6 |
| 36.975 | 13.3 |
| 37.618 | 17.4 |
| 37.799 | 19.7 |
| 38.242 | 16.2 |
| 38.882 | 15.5 |
| 39.432 | 8.1 |
| 39.577 | 9.0 |
| 40.198 | 18.4 |
| 41.451 | 8.2 |
| 42.109 | 14.6 |
| 42.816 | 8.7 |
| 43.969 | 11.5 |
| 44.213 | 14.2 |
| 44.812 | 11.9 |

1. A process for the preparation of a compound of formula (I): 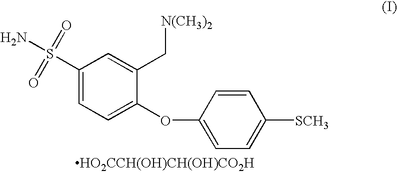
comprising the reaction of a compound of formula (VII) 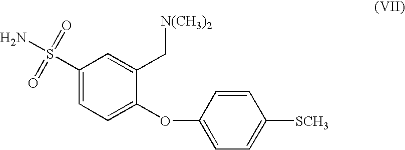
with tartaric acid in a suitable solvent.

2. A process according to claim 1 where the solvent is methyl ethyl ketone and the (L) form of tartaric acid is used.

3. A process according to claim 1 which further comprises the preparation of a compound of formula (VII) by reaction of a compound of formula (IX) 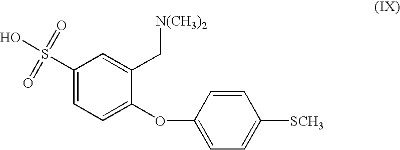
with a suitable chlorinating agent, in a suitable solvent, to generate a sulfonyl chloride in situ; and then quenching this sulfonyl chloride by addition of a suitable ammonia source to the reaction mixure.

4. A process according to claim 3 which further comprises the preparation of a compound of formula (IX) by the reaction of a compound of formula (VIII) 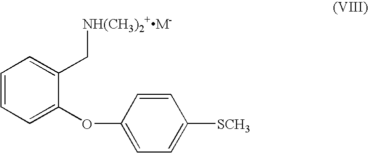
wherein M is a suitable counter-ion, with a suitable sulfonylating agent in a suitable solvent.

5. A process according to claim 4 where the solvent is methanesulfonic acid or trifluoroacetic acid and M is the chloride, hydrogensulfate, or methanesulfonate ion.

6. A process according to claim 4 which further comprises the preparation of a compound of formula (VIII) by the reaction of a compound of formula (IV) 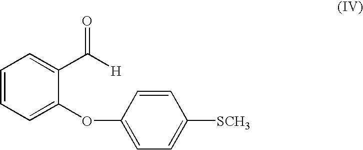
with a suitable dimethylamine source in the presence of a suitable reducing agent, in a suitable solvent and then treating the resultant amine with a suitable acid.

7. A process according to claim 6 where the reducing agent is formic acid and the solvent is N,N-dimethylformamide.

8. A process according to claim 6 which further comprises the preparation of a compound of formula (IV) by the reaction of a compound of formula (II) 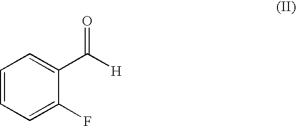
with a compound of formula (III) 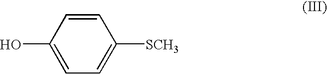
in the presence of a suitable base, in a suitable solvent.

9. A process, according to claim 8, for the preparation of a compound of formula (VIII) wherein:
(i) the reaction of compounds (II) and (III) is carried out in N,N-dimethylformamide in the presence a base;
(ii) the resultant crude reaction mixture containing compound (IV) is treated with formic acid and then allowed to react at elevated temperature;
(iii) the salt of the resulting amine product is formed by treatment with an appropriate acid.

10. A process for the preparation of a compound of formula (VII): 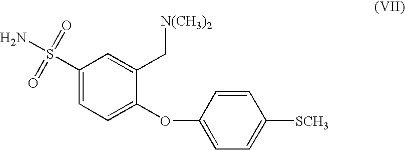
comprising reaction of a compound of formula (IX) 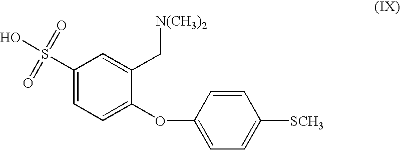
with a suitable chlorinating agent, in a suitable solvent, to generate a sulfonyl chloride in situ; and then quenching this sulfonyl chloride by addition of a suitable ammonia source to the reaction mixure.

11. A process for the preparation of a compound of formula (IX): 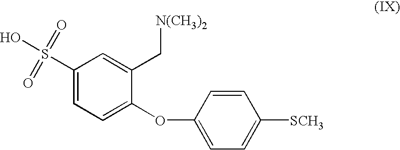
comprising the reaction of a compound of formula (VIII) 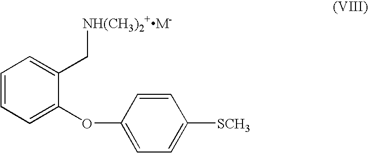
wherein M is the chloride, hydrogensulfate, or methanesulfonate ion, with a suitable sulfonylating agent in a methanesulfonic acid or trifluoroacetic acid as solvent.

12. A process for the preparation of a compound of formula (VIII) 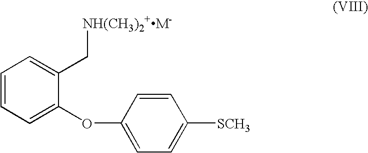
wherein M is a suitable counter-ion; comprising the reaction of a compound of formula (IV) 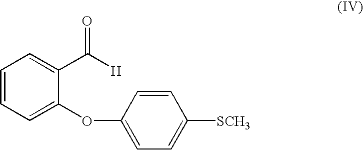
with a suitable dimethylamine source in the presence of a suitable reducing agent, in a suitable solvent and then treating the resultant amine with a suitable acid.

13. A compound of formula (IX):

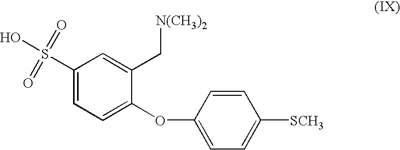

14. A compound of formula (VIII): 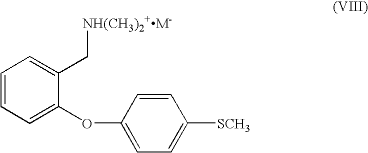
wherein M is the hydrogensulfate or methanesulfonate ion.
